(19) United States
(12) Patent Application Publication
(10) Pub. No.: US 2003/0104080 A1
(43) Pub. Date: Jun. 5, 2003

(54) Topical urea composition

(76) Inventors: Parashu Ram Singh, North York (CA); Alan Lorne Perlmutter, London (CA)

Correspondence Address: BLAKE, CASSELS & GRAYDON LLP, Box 25, Commerce Court West, 199 Bay Street, Toronto, ON, M5L 1A9, CA

(21) Appl. No.: 10/087,850

(22) Filed: Mar. 5, 2002

Related U.S. Application Data
(63) Continuation-in-part of application No. PCT/CA00/01031, filed on Sep. 7, 2000.
(60) Provisional application No. 60/152,637, filed on Sep. 7, 1999.

(51) Int. Cl. A61K 35/78; A61K 31/355; A61K 31/375; A61K 31/17

(52) U.S. Cl. 424/729; 514/458; 514/474; 514/588

(57) ABSTRACT

Topical composition that contains about 10 to about 50% by weight urea with respect to the total composition weight of the composition; and a topically effective amount of an anti-oxidant compatible with skin. Compositions containing vitamin E, vitamin C, vitamin D and green tea are described. Also described is a method of enhancing delivery of an anti-oxidant to the viable epidermis, including topically applying a composition of the invention to a skin surface of a mammal.

### RELATED APPLICATIONS

This application is a continuation-in-part application of PCT/CA 00/01031 filed Sep. 7, 2000 designating the United States, which application claims priority from U.S. Provisional Patent Application Serial No. 60/152,637 filed Sep. 7, 1999. Both of these prior applications are incorporated herein by reference. International patent application No. PCT/CA 00/01031 was published in English under Article 21 of the Patent Cooperation Treaty under No. WO 01/17484 on Mar. 15, 2001.

### FIELD OF THE INVENTION

This invention relates to a topical skin composition containing an active ingredient and urea for enhancing delivery of the ingredient. The active ingredient can be, alone, or in combination with another active ingredient, one or more of an antioxidant, vitamin, a β-glucan, or other active ingredient. Particularly useful is a composition containing vitamin E and urea, or a composition containing urea, vitamins A, C and E, and green tea extract.

### BACKGROUND OF THE INVENTION

Topical compositions are widely used in the cosmetics and pharmaceutical industries.

It is well known to include an active agent in a topical composition for the purpose of treating the skin. Such agents contribute to valuable effects, such as controlling ageing or pigmentation of the skin, promoting repair of damaged skin and contributing to skin cell renewal.

In terms of “active agents”, those relating to this invention include antioxidants, vitamins and β-glucans, and others listed below in connection with detailed embodiments. In the case of vitamins, some have antioxidant properties, and others are useful primarily for other beneficial effects. A particularly useful ingredient in the context of this invention is vitamin E.

It is known to topically apply vitamins for treatment of sunlight damage to skin^{1,2,3}, physical injury to skin^{4,5}, ageing of skin⁶, and pollution-challenged skin.⁷ Exposure to sunlight has been found to decrease amounts of antioxidants in skin.^{8,9} Vitamin C, topically administered in a moisturizing cream base, has been found to enhance the production of collagen^{10,11} which is involved in stimulation of fibroblasts necessary for the regeneration of older and damaged skin. Vitamin C administration has also been found to improve the lipid profile so as to enhance the barrier function of skin.¹² Topically applied vitamin C has also been found to have anti-inflammatory properties.¹³ There are many studies supporting the topical utility of certain vitamins.

Topically applied vitamin E is known to behave as an antioxidant^{14,15,16,17} and can serve to decrease healing time¹⁸ with reduction of the severity and frequency of pathological damage to cells.¹⁹ Vitamin E has been shown to enhance the ability of skin to retain moisture.²⁰ A topically applied mixture of vitamin E and vitamin C was shown to protect against sun damage to the skin.²¹ There is evidence vitamins E and C can protect the skin against oxidative damage caused by free radicals.²²

Vitamin A has also been found to offer protection of the skin from chemical insult,²³ but it should be transported through the skin to effectively promote the metabolism of skin cells.²⁴ Topically applied vitamin A palmitate has been found to improve skin elasticity.²⁵ Vitamin A also contributes to repair of photo-damaged skin by stimulating growth of the collagenous matrix.²⁶

Vitamins A, C and E are utilized by the body in defence against skin damage²⁷ and it has been suggested that the three vitamins are most effective together.²⁸ There are studies which suggest that vitamins C and E need to be present before sun damage occurs in order to be an effective defence thereagainst.^{29,30,31,32,33,34} Studies have shown, however, that oral administration of vitamin C and vitamin E does not lead to appreciably increased amounts of the vitamin in the skin.^{35,36}

The salutary effects of green tea are coming to be known. Green tea has been shown to counter the irritative effects of α-hydroxy acids and been found to be a potent antioxidant^{37,38}, to contain free radical scavengers³⁹, and to be a chemopreventive agent against skin tumors and solar radiation damage.^{40,41,42} It has been shown that green tea antioxidants can protect against general free radical damage and skin cancer in animal models.⁴³

β-glucans are high molecular weight phosphorylated polysaccharides, generally obtained from oats, which can be solubilized and used as moisturizers and also aid in the healing of wounds and infections.⁴⁴

U.S. Pat. No. 5,935,588, which issued Aug. 10, 1999 to Afriat et al., offers a recent example from the patent literature of a topical composition which can potentially include an active agent such as green tea, vitamin C and/or vitamin A. The specification of this patent describes an emulsion composition containing a water-sensitive active agent. The activity of water in an aqueous phase of the emulsion is lowered to 0.85 or less by inclusion of a polyol so as to stabilize the water-sensitve agent against degradation. The active agent or agents can be used in the composition in an amount ranging from 0.001 to 15% by weight, preferably from 0.01 to 10% and more preferably from 0.05 to 5% by weight with respect to the total weight of the composition. In specific embodiments, the water-sensitive agent is an enzyme sold under the tradename Subtilisine SP544 present in the amount of 0.1%. The specification states that other water-sensitive active agents include green tea, ascorbic acid, vitamin A and urea, but describes no specific embodiment involving any of these agents.

U.S. Pat. No. 5,935,994, which issued Aug. 10, 1999 to Nimni, describes a topical composition containing Vitamins A and E and including an organic penetrant. Such organic penetrants include lower alkyl diols, C₁₀ to C₂₀ fatty acids and esters thereof, and C₄ to C₂₀ aliphatic alcohols. Exemplary of such penetrants are propylene glycol, oleic acid, butyl alcohol and, preferably, benzyl alcohol. Generally speaking, the amount of penetrant is suggested to vary between about 0.5 and about 10 weight percent.

U.S. Pat. No. 5,874,074, which issued to Smith on Feb. 23, 1999, describes a topical lotion containing a therapeutic agent which can be a dermatological agent such as a vitamin A derivative and a penetration enhancer. Particular enhancers that are said to be useful in the lotions include dimethyl sulfoxide, N,N-dimethyl acetamide, 2-pyrrolidone, 1-methyl-2-pyrrolidone, Carbitol solvent (Union Carbide), propylene carbonate, 1,5-dimethyl-2-pyrrolidone, 2-pyrrolidone-5-carboxylic acid, and the like, wherein the lotion includes a penetration enhancing agent in an amount of about 0.01 to 20 weight percent. Among many other ingredients, it is also suggested that the dermatological agent could also be an anti-psoriatic compound such as anthralin (dithranol), coal tar extract, and the like; a keratolytic agent such as salicylic acid, urea, and the like.

One will find, in reviewing the literature relating to topical compositions, that such compositions generally include a variety of types of ingredients, each for their own purpose(s). Thus, a person skilled in the art, generally includes many such ingredients as taught in the prior art. U.S. Pat. No. 5,741,499, for example, which issued to Arnauld et al. Apr. 21, 1998, describes a homogeneous composition for use in cosmetics and dermatology which includes an organic fluorinated compound and provides a fair description of the types of ingredients and examples of such that can be included in topical compositions. Along this vein, U.S. Pat. No. 5,935,585, which issued to Bernardon et al on Aug. 10, 1999, describes topical pharmaceutical and cosmetic compositions containing biaromatic compounds. It is suggested to include in the compositions various combinations of a retinoid compound, a D vitamin, an anti-free radical agent, an α-hydroxy acid, an ion channel blocker, a moisturizing agent, a wetting agent, a depigmenting agent, an antiseborrhoeic or antiacne agent, an antibiotic, an antifungal agent, a hair regrowth promoter, a non-steroidal anti-inflammatory agent, or an anti-psoriatic agent, 5,8,11,14-eicosatetraynoic or 5,8,11-eicosatrynoic acid or ester or amide thereof.

Nonetheless, the inventors herein have invented what appears to be a new composition for the purpose of improved topical delivery of active ingredients, particularly vitamins, antioxidants and β-glucans that are of benefit to the skin and most particular vitamin E.

### SUMMARY OF THE INVENTION

In a broad aspect, the present invention is a topical composition containing urea and one or more active ingredient(s), in which the urea is present in the composition in an amount sufficient to enhance penetration of the active ingredient(s) of the composition. The invention includes a method of treatment of living skin (the viable epidermis, below the stratum corneum, and dermis) enhancing delivery of active ingredient(s) thereto by topically applying to the skin surface a composition of the invention.

A preferred active ingredient is vitamin E and a preferred combination of ingredients is vitamins A, C and E, and green tea. Other active ingredients are antioxidants such as retinyl palmitate, β-carotene, tocopherol acetate, ascorbic acid, green tea, black tea, quercetin (flavonoids), sea kelp, pycnogenols (proanthocyanidins), selenium and alkylgylcerol-AKG (shark liver oil), taken alone or in combination, and others described below in connection with the detailed embodiments.

An amount of urea sufficient to enhance penetration of the antioxidant is determined for each ingredient or family of ingredients for inclusion in a single composition of the invention. This is generally in excess of 10% and up to about 50% urea by weight of the total composition.

Other agents that are typically included in topical compositions can be included in compositions of the invention, and are described in connection with detailed embodiments.

Compositions of the invention are generally used in situations in which it would be found advantageous to have the active ingredient(s) delivered to living skin below the stratum corneum. Compositions of the present invention can find usefulness in application to skin of subjects suffering from diabetes, menopause, eczema, scleroderma, psoriasis, cancer, multiple sclerosis, allergy sensitivities, Down's syndrome, circulatory disorders, and so on.

In a particular aspect, the invention is a topical composition that includes about 10 to about 50% by weight urea with respect to the total composition weight of the composition; and a topically effective amount of an anti-oxidant compatible with skin. the anti-oxidant can be selected from the group consisting of vitamin E, vitamin C, vitamin D, retinyl palmitate, β-carotene, green tea, black tea, quercetin, sea kelp, pycnogenols (proanthocyanidins), selenium and alkylgylcerol-AKG, allopurinol, α-lipoic acid, astaxanthin, azulenic retinoid compounds, coenzyme Q-10, cysteine, zinc, copper, magnesium, potassium, selenium, BHA, BHT, melatonin; N-acetylcysteine, and combinations thereof.

Preferably, the composition includes between about 10% and about 45% urea, about 15% and about 40% about urea, between about 20% and about 40% urea, between about 20% and about 35% urea, between about 20% and about 30% urea, particularly, about 21%, aobut 22%, about 23%, about 24%, about 25%, about 26%, about 27%, about 28%, about 29%, or about 30% urea.

A preferred anti-oxidant is vitamin E, which can be in the form of tocopherol acetate. The composition can include up to about 10% by weight of vitamin E, but more preferably, about 5% and at least about 0.1% by weight of vitamin E. Other compositions include at least about 0.5%, or at least about 1% be weight of vitamin E, between about 1% and about 4%, between about 1% and 2% by weight of vitamin E, or about 1% by weight of vitamin E, or about 1.5%.

A preferred family of anti-oxidants present in a composition is vitamin A, vitamin C, vitamin E and green tea extract, although these ingredients can be taken separately or in any combination. Preferably, the entire family is present in the composition.

Such a composition can include up to about 5% by weight of vitamin A, at least about 0.1%, between about 0.2% and 4%, between about 0.3% and 3%, or about 0.3% by weight of vitamin A. The composition can include up to about 10% by weight of vitamin C, or at least about 0.1%, between about 0.1% and 5%, between about 0.1% and about 3%, between about 0.1 and 2%, between about 0.1% and 1%, or about 0.1% or about 0.5% vitamin C. The composition can include up to about 10% by weight of green tea extract. 37. The composition of any of claims 21 to 36, comprising at least about 0.1% by weight of green tea extract, between about 0.1% and 5%, between about 0.1% and 3%, between about 0.1% and 1%, or about 0.3%, or about 0.5% by weight of green tea extract.

In another aspect, the invention is a method of enhancing delivery of an anti-oxidant to the viable epidermis, the method comprising the step of topically applying a composition of the invention to a skin surface of a mammal.

In another aspect, the invention is the use of a composition of the invention in the preparation of a topical medicament for use in delivery of one or more anti-oxidants to the viable epidermis of a mammal.

In another aspect, the invention includes topical use of a composition of the invention in the delivery of one or more anti-oxidants to the viable epidermis of a mammal.

The invention also includes a method of manufacturing a topical preparation comprising a composition of the invention, the method comprising combining the one or more anti-oxidants and urea so as to form a homogeneous topical skin preparation. Preferably, the skin preparation is a cosmetic preparation. The method can include incorporating water into the preparation, wherein the final amount of water is between about 15% and 80% by weight, between about 30% and 80%, or between about 50% and 70%. The method can further include incorporating glycerin into the preparation, wherein the final amount of glycerin is up to about 20% by weight, but more preferably the amount of glycerin is between about 55 and 15%.

### DESCRIPTION OF THE DRAWINGS

FIGS. 1(a) to 1(e) graphically illustrate the effects of a composition containing 15% urea and vitamins A, C, and E, and green tea extract (dashed line), and a similar composition without any of vitamins A, C, and E, and green tea extract (solid line) on the condition of skin as measured using a Corneometer, as evaluated for five different subjects. The condition of an untreated area (thickened line) was also evaluated. Measurements were taken over a 10 ½ day period as explained in detail below.

### DESCRIPTION OF DETAILED EMBODIMENTS

The present invention is a topical composition that includes at least one active ingredient in combination with urea in which the urea is present in an amount sufficient to enhance penetration of the active ingredient(s).

The active ingredient(s) is selected for its beneficial effects to the skin, which effect is to be enhanced by exposure of the viable epidermis and/or dermis, which underlies the stratum corneum, to the ingredient. By including urea in a concentration sufficient to enhance penetration of the agent through the stratum corneum in a topical composition, such effect is enhanced. Generally speaking, the proportion of the composition which should be urea is at least 10 percent by weight. (100 gm of a composition that is 10 percent by weight of a component would contain 10 gm of that component.) Because of the enhanced penetration, it is possible to devise compositions containing less of a given active ingredient than would be necessary to obtain the same or comparable effect due to the the ingredient's activity in the absence of a penetration enhancing amount of urea.

The precise minimum amount of urea necessary to enhance penetration of a particular active ingredient agent(s) is determined according to the method given below. In general, the minimum amount of urea that is necessary to obtain penetration enhancement is at least about 10%, but it would generally be higher, and could be as high as 50%. Typical compositions have about 15%, or about 20%, or about 25%, or about 30%, or about 35%, or about 40% urea. In the context of this invention, percentages are given as “weight percent”.

A preferred active ingredient is selected from vitamins, antioxidants and β-glucans.

Preferred vitamins are vitamin E (particularly, alpha tocopherol, as well as beta, delta, and gamma-tocopherols and alpha, beta, delta and gamma tocotrienols), vitamin C, and vitamin D. Of course, certain vitamins, such as vitamin E, are known to have antioxidant properties.

Preferred antioxidants include retinyl palmitate, β-carotene, tocopherol acetate, ascorbic acid, green tea, black tea, quercetin (flavonoids), sea kelp, pycnogenols (proanthocyanidins), selenium and alkylgylcerol-AKG (shark liver oil).

It is generally considered that if β-carotene is included, vitamin E should also be included as discolorization of the skin by vitamin A can be reduced in the presence of vitamin E.⁴⁵

The beneficial effects of certain vitamins, as far as the skin is concerned, are known. For example, it has been shown that vitamins E and C can protect the skin against oxidative damage caused by free radicals.⁴⁶

Green tea is known to contain polyphenols and their use in antioxidant formulations has been suggested. See, for example, U.S. Pat. No. 5,648,377, which issued to Bombardelli et al on Jul. 15, 1997.

Other antioxidants include: Allopurinol; α-lipoic acid; astaxanthin; azulenic retinoid compounds; vitamin A related compounds such as β-carotene, carotenoid, lycopene, xanthophylls and lycopene; coenzyme Q-10; cysteine; metals such as zinc, copper, magnesium, potassium selenium; BHA; BHT; maharishi amrit kalash (MAK); melatonin; N-acetylcysteine (NAC); olive oil; phenolics; pyrimidines; activin (from seeds of red grapes); superoxide dismutase (SOD); prozyme (Polbax); black tea in addition to green tea (camelia sinensis); proanthocynidins (OPC); pycnogenol grape seed); curcumin from tumeric; silymarin, the flavonoid complex of milk thistle (Silybum marianum); cat's claw; ginkgo biloba; silica hydride.

Urea is a well known component of topical compositions. In the bulk of products in which is it used, it is included as a moisturizing agent. This is true, for example, where it is suggested for use in compositions described in U.S. Pat. No. 5,935,585. When used as a moisturizing agent in a composition, the amount of urea included is usually limited to small amounts.

Urea is also known to improve the elasticity of the stratum corneum.⁴⁷

The influence of urea in topical compositions on penetration of other ingredients, such as hydrocortisone has been described in the past.⁴⁸

Use of urea in connection with analgesics is also known. U.S. Pat. No. 5,814,659, which issued to Elden on Sep. 29, 1998, (Canadian Patent Application No. 2,203,456 laid open Oct. 23, 1997) describes the use of a chaotropic agent, particularly urea, in combination with an analgesic agent, particularly lidocaine. A product containing these ingredients is currently on sale in the United States under the name Lespain. In this context 10% urea is used.

Studies of the penetration enhancing abilities of urea have been conducted.^{49,50,51}

Preliminary results have been obtained by the inventors. During a one-week period, three lotions were applied by a single subject to different skin areas to be equally exposed to the sun. Lotion A contained 2% by weight lactic acid and 0.2% by weight malic acid in an oil-in-water emulsion. Lotion B was the same as lotion A with urea added to make up 10% by weight of the total composition. Lotion C was the same as lotion B with 0.2% allantoin, 0.3% vitamin E, 0.25% vitamin A, 0.10% vitamin C, and 0.3% green tea extract, all percentages being weight percent.

The three areas of the subject were equally exposed to natural sunlight over a one week period. Results were evaluated by measuring skin impedance using a Surface Characterizing Impedance Monitor (SCIM) developed by Ollmar, which measures bioelectrical impedance of the skin at multiple frequencies.^{52,53,54,55,56,57} The instrument is basically an AC-bridge fabricated from standard laboratory instruments: a function generator, a digital oscilloscope, impedance references, and a driver for the probe.

The results obtained are given in Table I.

TABLE I

| | Impedance | | Lotion A | Lotion B | Lotion C |
|---|---|---|---|---|---|
| Depth | Before | After | Change | Change | Change |
| 1 | 13.1559 | 13.4228 | 0.2669 | 0.2113 | −0.0823 |
| 2 | 13.0085 | 13.1488 | 0.1403 | 0.7589 | −0.0428 |
| 3 | 12.716 | 13.7932 | 1.0772 | 0.4344 | −0.0582 |
| 4 | 12.0382 | 12.6122 | 0.574 | 0.4727 | 0.2795 |
| 5 | 11.7776 | 12.4459 | 0.6683 | 0.4298 | 0.035 |

The baseline is given as the “before” measurement in Table I. The depths of skin measured varied from the most superficial stratum corneum (1) to the live epidermis (5).

Electrical impedance of skin is less for intact, more hydrated skin. The results given in Table I thus indicate that, while urea itself provides some benefit beyond the components of lotion A, the combined effect of urea and vitamin E, or urea and vitamin A, or urea and vitamin C or urea and green tea extract greatly exceeds that of urea alone.

The results obtained, although obtained in feasibility tests to establish effectiveness of a composition containing urea in combination with each of these agents in improving skin condition, are surprising in that the concentration of agent which obtains the result is very small.

Particular compositions containing vitamins A, C, E and green tea extract are contemplated as follows:

Composition A:

| INGREDIENT | % (w/w) |
|---|---|
| Water | 43.65 |
| Urea | 25 |
| PEG-100 Stearate | 6 |
| Beeswax | 5 |
| Mineral oil | 5 |
| Lanolin | 4 |
| Cetyl Alcohol | 3 |
| Triethanolamine | 2.5 |
| Lactic acid | 2 |
| Silk Amino Acid | 1 |
| Imidazolidinyl urea | 0.4 |
| Green Tea Extract | 0.3 |
| Tocopheryl Acetate | 1 |
| Retinyl Palmitate | 0.25 |
| Malic Acid | 0.2 |
| Methyl Paraben | 0.2 |
| Allantoin | 0.2 |
| Ascorbic Acid | 0.1 |
| Propyl Paraben | 0.1 |
| Trisodium EDTA | 0.1 |
| | 100 |

Composition A was obtained according to the following procedure:

% (W/W)

PHASE I (aqueous phase)

| | |
|---|---|
| Water | 43.65 |
| Urea | 25 |
| Imidazolidinyl urea | 0.4 |
| Methyl paraben | 0.2 |
| Malic acid | 0.2 |
| Allantoin | 0.2 |
| Ascorbic acid | 0.1 |
| Trisodium EDTA | 0.1 |

PHASE II (oil phase)

| | |
|---|---|
| PEG-100 stearate | 6 |
| Beeswax | 5 |
| Mineral oil | 5 |
| Lanolin | 4 |
| Cetyl alcohol | 3 |
| Tocopheryl acetate | 0.3 |
| Propyl paraben | 0.1 |

PHASE III

| | |
|---|---|
| Triethanolamine | 2.5 |

PHASE IV

| | |
|---|---|
| Lactic acid | 2 |

PHASE V

| | |
|---|---|
| silk amino acid | 1 |
| Green tea extract | 0.3 |
| Retinyl palmitate | 0.25 |

In a s.s. kettle the ingredients of phase I are combined and heated to 70°-75° C. and maintained at that temperature. In a separate s.s. kettle the ingredients of phase II are combined and heated to 75°-80° C. and maintained at that temperature. Phase II is added at 75°-80° C. to phase I at 70°-75° C. with mixing to homogenous solution and the solution is permitted to cool. At 60-65° C., phase III is added and cooling and mixing is continued. At 50°-55° C., phase IV is added and cooling and mixing is continued. At 35°-40° C., the ingredients of phase V are added with mixing. Cooling and mixing until a temperature of 30°-35° C. is reached and mixing is stopped.

Composition B:

| INGREDIENT | %, W/W |
|---|---|
| Water | 46.75 |
| Urea | 20 |
| PEG-100 Stearate | 6 |
| Cetyl Alcohol | 6 |
| Beeswax | 4 |
| Isopropyl Myristate | 4 |
| Triethanolamine | 2.5 |
| Emulsifying Wax | 2 |
| Lactic Acid | 2 |
| Petrolatum | 2 |
| Mineral oil | 1 |
| Silk Amino Acid | 1 |
| Imidazolidinyl Urea | 0.3 |
| Green Tea Extract | 0.3 |
| Tocopheryl Acetate | 1 |
| Retmyl Palmitate | 0.25 |
| Methyl Paraben | 0.2 |
| Allantoin | 0.2 |
| Malic Acid | 0.2 |
| Propyl Paraben | 0.1 |
| Ascorbic Acid | 0.1 |
| Trisodium EDTA | 0.1 |
| | 100 |

Composition B was obtained according to the following procedure:

% (W/W)

PHASE I (aqueous phase)

| | |
|---|---|
| Water | 46.75 |
| Urea | 20 |
| Imidazolidinyl urea | 0.3 |
| Methyl paraben | 0.2 |
| Allantoin | 0.2 |
| Malic acid | 0.2 |
| Ascorbic acid | 0.1 |
| Trisodium EDTA | 0.1 |

PHASE II (oil phase)

| | |
|---|---|
| PEG-100 stearate | 6 |
| Cetyl alcohol | 6 |
| Beeswax | 4 |
| Isopropyl myristate | 4 |
| Emulsifying wax | 2 |
| Petrolatum | 2 |
| Mineral oil | 1 |
| Tocopheryl acetate | 1 |
| Propyl paraben | 0.1 |

PHASE III

| | |
|---|---|
| Triethanolamine | 2.5 |

PHASE IV

| | |
|---|---|
| Lactic acid | 2 |

PHASE V

| | |
|---|---|
| silk amino acid | 1 |
| Green tea extract | 0.3 |
| Retinyl palmitate | 0.25 |

In a s.s. kettle the ingredients of phase I are combined and heated to 70°-75° C. and the temperature maintained. In a second s.s. kettle the ingredients of phase II are combined and heated to 75°-80° C. and the temperature maintained. Phase II, at 75°-80° C. is added to phase I at 70°-75° C. with mixing. A homogeneous solution is obtained and the mixture is permitted to cool with mixing. At 60°-65° C., phase III is added to the solution and cooling and mixing is continued. At 50°-55° C., phase IV is added and mixing and cooling is continued.

At 35°-40° C., ingredients of phase V are added in the order indicated with mixing and mixing is continued until the solution reaches a temperture of 35°-35° C.

Composition C:

| INGREDIENT | % (W/W) |
|---|---|
| Water | 58.55 |
| Urea | 15 |
| Glycerin | 6 |
| PEG-100 Stearate | 3.5 |
| Emulsifying Wax | 3 |
| Squalene | 3 |
| Cetyl Alcohol | 2.5 |
| Triethanolamine | 2.5 |
| Lactic Acid | 2 |
| Silk Amino Acid | 1 |
| Imidazolidinyl Urea | 0.4 |
| Tocopheryl Acetate | 1 |
| Green Tea Extract | 0.3 |
| Retinyl Palmitate | 0.25 |
| Methyl Paraben | 0.2 |
| Allantoin | 0.2 |
| Malic Acid | 0.2 |
| Propyl Paraben | 0.1 |
| Ascorbic Acid | 0.1 |
| Trisodium EDTA | 0.1 |
| Carbomer 934P | 0.1 |
| | 100 |

Composition C was obtained according to the following procedure:

% (W/W)

PHASE I (aqueous phase)

| | |
|---|---|
| Water | 58.55 |
| Carbomer 934 P | 0.1 |
| Urea | 15 |
| Imidazolidinyl urea | 0.4 |
| Methylparaben | 0.2 |
| Allantoin | 0.2 |
| Ascorbic acid | 0.1 |
| Malic acid | 0.2 |
| Trisodium EDTA | 0.1 |
| Glycerin | 6 |

PHASE II (oil phase)

| | |
|---|---|
| PEG-100 stearate | 3.5 |
| Emulsifying wax | 3 |
| Squalane | 3 |
| Cetyl alcohol | 2.5 |
| Tocopheryl acetate | 1 |
| Propyl paraben | 0.1 |

PHASE III

| | |
|---|---|
| Triethanolamine | 2.5 |

PHASE IV

| | |
|---|---|
| Lactic acid | 2 |

PHASE V

| | |
|---|---|
| Silk amino acid | 1 |
| Green tea extract | 0.3 |
| Retinyl palmitate | 0.25 |

In a s.s. kettle the ingredients of phase I are added the order indicated and mixed until the carborner is completely dispersed and hydrated. The solution is headed to 70°-75° C. and the temperature maintained. In a separate s.s. kettle the ingredients of phase II are combined and heated to 75°-80° C. with mixing and the temperature maintained. The phase II mixture at 75°-80° C. is added to the phase I solution at 70°-75° C. with mixing. The batch is mixted to obtain a homogeneous solution and the solution is permitted to cool. At 60°-65° C., phase III is added with mixing and cooling is continued. At 50°-55° C., phase IV is added with mixting and cooling continued. At 35°-40° C., the ingredients of phase V are added with mixing in the order indicated. Mixing is stopped when a temperature of 30°-35° C. is reached.

Composition D:

| INGREDIENT | % (W/W) |
|---|---|
| Water | 63.25 |
| Urea | 10 |
| Propylene Glycol | 5 |
| Squalene | 4.5 |
| Isopropyl Myristate | 4 |
| Triethanolamine | 2.5 |
| PEG-100 Stearate | 2 |
| Lactic Acid | 2 |
| Cetyl Alcohol | 2 |
| Emulsifying Wax | 1 |
| Silk Amino Acid | 1 |
| Imidazolidinyl Urea | 0.4 |
| Green Tea Extract | 0.3 |
| Tocopheryl Acetate | 1 |
| Retinyl Palmitate | 0.25 |
| Allantoin | 0.2 |
| Malic Acid | 0.2 |
| Ascorbic Acid | 0.1 |
| Quaternium - 15 | 0.1 |
| Trisodium EDTA | 0.1 |
| Carbomer 941 | 0.1 |
| | 100 |

Composition D was obtained according to the following procedure:

% (W/W)

PHASE I (aqueous phase)

| | |
|---|---|
| Water | 62.75 |
| Carbomer 941 | 0.1 |
| Urea | 10 |
| Imidazolidinyl urea | 0.4 |
| Allantoin | 0.2 |
| Ascorbic acid | 0.1 |
| Malic acid | 0.2 |
| Trisodium EDTA | 0.1 |
| Propylene glycol | 5 |

PHASE II (oil phase)

| | |
|---|---|
| Squalane | 4.5 |
| Isopropyl myristate | 4 |
| PEG-100 stearate | 2 |
| Emulsifying wax | 1 |
| Cetyl alcohol | 2 |
| Tocopheryl acetate | 1 |

PHASE III

| | |
|---|---|
| Triethanolamine | 2.5 |

PHASE IV

| | |
|---|---|
| Lactic acid | 2 |

PHASE V

| | |
|---|---|
| Water | 0.5 |
| Quaternium-15 | 0.1 |

PHASE VI

| | |
|---|---|
| Silk Amino acid | 1 |
| Green tea extract | 0.3 |
| Retinyl palinitate | 0.25 |

In a s.s. kettle, the ingredients of phase I are combined in the order indicated and mixed until carborner is completely dispersed and hydrated. The mixturue is heated to 70°-75° C. and the temperature maintained. In a separate s.s. kettle the ingredients of phase II are combined and heated to 75°-80° C. with mixing and the temperature maintained. The phase II mixture is added at 75°-80° C. to phase I at 70°-75° C. with mixing. The mixture is mixed to obtain a homogeneous solution and permitted to cool with mixing. At 60°-65° C., phase III is added to the batch and mixing and cooling are continued. At 50°-55° C., phase IV is added to the batch and mixing and cooling are continued. At 45°-50° C., phase V is added to the batch and mixing and cooling is continued. At 35°-40° C., the ingredients of phase VI are added to the batch in the order indicated and mixing is continued until a temperature of 30°-35° C. is reached.

It is preferable to have homogeneous formulations.

In vitro skin penetration studies can be used to evaluate a suitable amount of urea to be used in connection with a particular ingredient. The penetration of vitamins through human skin can be measured using various formulations with and without urea.

Transepidermal water loss (TEWL) can be measured by evaporimetery using a Servo-Med evaporimeter or similar device, for example.

In vivo dermatopharmacokinetic studies can be carried out to determine the effects of urea on penetration enhancement of active ingredients. Test compositions including an ingredient to be evaulated are prepared along with control compositions, which are the same except that the urea is omitted. Each composition is applied to the skin surface at 2 mg/square cm for between about 0.5 to 6 hours. After the specified period of time the site is washed thoroughly with mild detergent and water. The site is dried. The site is then tape stripped using D-Squame adhesive disks. Five strips are taken and combined and then five more and five more for a total of 25. The tapes are then extracted and analyzed for the active ingredient, say vitamin E, using HPLC. Higher levels of vitamin E in the lower tape strips from sites to which urea-containing compositions were applied indicate a positve effect on vitamin penetration.

In vivo skin penetration studies can also be carried out using dermatopharmacokinetics using tape stripping in order to determine a suitable amount of urea to be included in a composition in combination with a particular active ingredient. Studies similar to the foregoing are carried out for a number of concentrations of urea, say varying from 10 to 50%, at increments of 5%.

In a feasibility study, Composition E, containing 25% urea and vitamin E was tested. The formulations of compositions used in the studies are as follows:

Ingredients (w/w %)

| | Control | Composition E |
|---|---|---|
| Propyl paraben | 0.1 | 0.1 |
| Tetra sodium EDTA | 0.1 | 0.1 |
| Methyl paraben | 0.2 | 0.2 |
| Triethanolamine 99% | 0.25 | 0.25 |
| Imidazolidinyl urea | 0.4 | 0.4 |
| Silk protein (amino acid) | 2 | 2 |
| Cetyl alcohol | 3 | 3 |
| Lactic acid | 3 | 3 |
| Malic acid | 3 | 3 |
| Lanolin | 4 | 4 |
| Beeswax (synthetic) | 5 | 5 |
| Mineral oil—medium | 5 | 5 |
| Vitamin B (tocopheryl acetate) | 5 | 5 |
| GMS/peg 100 stearate | 6 | 6 |
| Urea USP | 0 | 25 |
| Deionized water | 62.95 | 37.95 |

In this case, 2.0 mg/cm of the composition was applied to 5 cm² of the forearm. After 120 minutes, each of the test areas was rinsed with warm water for 20 seconds. The disks were extracted with acetone:chloroform (50:50) with 10 minutes of sonication and then the residue was extracted again with 5 minutes of sonication. The extracts were dried under N₂ and then resuspended with ethanol. The ethanol solution was filtered through a nylon syringe filter. An aliquot was analyzed by HPLC.

The vitamin E (vitamin E acetate) was determined using an isocratic HPLC method. The wavelength was 290 nm, mobile phase was methanol, and the flow rate was 0.9 ml/min. A 20-μl sample was injected into the HPLC. Retention time was 4.3 min or 5.4 min for vitamin E and vitamin E acetate, respectively. The linear range was: 0.002036-0.3054 mg/ml (r²=0.9997).

The results shown in Table One, are given as the area under the peak from the HPLC analysis. Statistical analysis was performed using 2 tailed paired t. The level of vitamin E was found to be higher for the composition containing urea and vitamin E but because of the small sample size and high variability the results were only statistically significant at the deepest level.

TABLE ONE

| | Level 1 (Tapes 2-6) | | | Level 2 (Tapes 7-11) | | | Level 3 (Tapes 12-16) | | |
|---|---|---|---|---|---|---|---|---|---|
| Subject | Urea | Control | Difference | Urea | Control | Difference | Urea | Control | Difference |
| 1 | 6937 | 3452 | 3485 | 1785 | 789 | 996 | 2400 | 1272 | 1128 |
| 2 | 1506 | 903 | 603 | 719 | 0 | 719 | 622 | 0 | 622 |
| 3 | 5445 | 5396 | 49 | 4780 | 2888 | 1892 | 2640 | 1306 | 1334 |
| Average | 4629 | 3250 | 1379 | 2428 | 1226 | 1202 | 1887 | 859 | 1028 |
| T test | | | | | | | | | |
| P value | | | 0.32 | | | 0.08 | | | 0.04 |

Also observed was that the percent increase in vitamin E, comparing urea to the control, was greatest at the lowest level tested:

| Level | Urea | Control | Percent Increase |
|---|---|---|---|
| 1 | 4629 | 3250 | 42 |
| 2 | 2428 | 1226 | 98 |
| 3 | 1887 | 859 | 120 |

These results establish that urea is effective in enhancing delivery of vitamin E to the viable epidermis.

An especially preferred composition of the present invention thus is one that contains between 10 and 50% urea, more preferably between 15 and 50% urea, more preferably between 20 and 45% urea, and more preferably still between 25 and 40%, and most preferrably about 25%.

Further feasibility studies were conducted using a formula containing 15% urea, Composition F. Composition F had the same composition as that set out above for Composition C. In these studies, five subjects were tested over a period of 10 ½ days. For each subject, three different areas of skin were tested. The first area was not treated. The second area was treated with a control containing urea but no vitamin E, here called Composition F′. The third area was treated with Composition F.

Composition F′:

| Ingredient | % (w/w) |
|---|---|
| Carbomer 934P | 0.1 |
| Cetyl alcohol | 2.5 |
| Deionized water | 56.1 |
| Emulsifying wax NF (polawax) | 3 |
| Glycerin | 6 |
| GMS/peg 100 stearate | 3.5 |
| Hydrogenated polyisobutene | 3 |
| Imidazolidinyl urea | 0.4 |
| Lactic acid | 3 |
| Malic acid | 2 |
| Methyl paraben | 0.2 |
| Propyl paraben | 0.1 |
| Silk protein (amino acid) | 1 |
| Tetra sodium EDTA | 0.1 |
| Triethanolamine 99% | 4 |
| Urea USP | 15 |

The effects of the two treatments relative to the non-treated area and to each other were followed using a Corneometer (COURAGE+KHAZAKA electronic GmbH, Mathias-Brüggen-Str. 91, D-50829 Köln-Germany, **www.courage-khazaka.de**), which provides a measurement of skin moisture based on a capacitance method. The measurement is based on the different dielectric constant of water and other substances. The measuring capacitor shows changes of capacitance according to the moisture content. Used according to the manufacturer's protocol, there is a probe which is touched to the skin for about a second to take a given measurement.

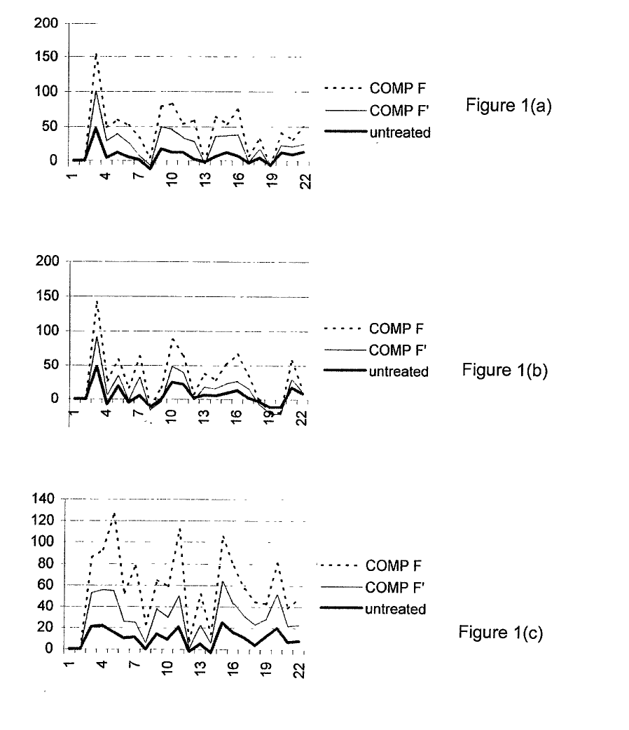

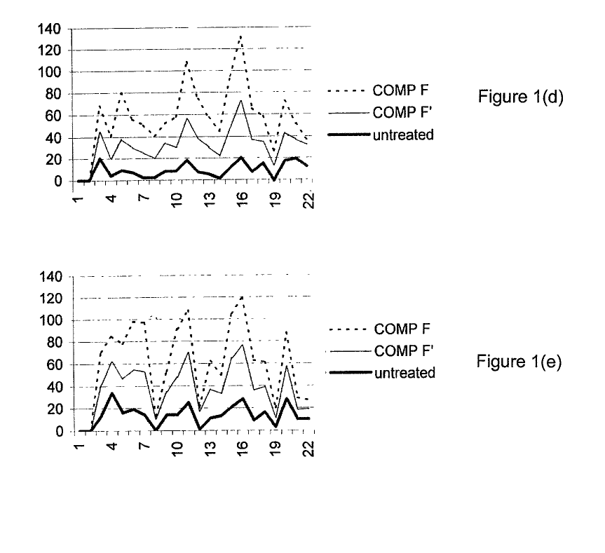

To begin, Compositions F and F′, were applied to the two treatment areas. The compositions were similarly applied and measurements taken 12 hours later and at 12 hour intervals thereafter. Treatment with Compositions F and F′ were stopped after the eighth day (i.e., after the 16th treatment) but measurements were continued, for a total of twenty-two measurements. Results are plotted in FIGS. 1(a) to 1(e), one plot for each of the five subjects. The plot above the most lightly shaded area in each figure is that obtained by taking measurement using the Corneometer of the area treated with Composition F. The initial measurement taken of a particular area was substracted from each measurement before plotting of the results. The plot above the most darkly shaded area in each figure is that obtained from measurements taken of the area treated with Composition F′. The remaining plot shows measurements obtained from the untreated area.

The x-coordinate of each plot shows the number of the measurement taken, at 12-hour intervals. The y-coordinate is the reading taken from the Corneometer having the initial reading for the area subtracted. The higher the reading the greater the moisture content of the skin.

As can be seen from the plots of FIGS. 1(a) to 1(e), although changes in moisture content vary in about the same direction from site to site of a given subject, the plot obtained from the area treated with Composition F is generally higher than that obtained from Composition F′, which is in turn generally higher than the plot obtained from the untreated area.

Further, it can be seen that the salutary effect continued for the three day test period after cessation of treatment.

There is mention in the art of the penetration enhancing properties of urea in connection with various active substances. For example, Wohlrab states that the promotion of drug penetration by urea can be exploited so as to improve therapeutic efficacy at the same concentration of an active substance and to achieve the same therapeutic efficacy with a considerably lower concentration. With respect to hydrocortisone, it has been shown that when low concentrations of urea are used, enhancement of penetration is barely apparent, whereas with a urea content of between 5% and 10% there is a particular increase, but apparently, when the urea concentrations are raised further, no further decisive changes are detectable.

Raab also suggests that urea increases the bioavailablity, or topical activity, of other drugs. In addition to the combination of glucocorticoids, e.g., 1% hydrocortisone with 10% urea, Raab describes improvement in the antisporiatic action of anthralin in a composition containing 17% urea. Raab also describes treatment of certain severe ichthyoses with a combination of 0.03% tretinoin (all-trans-retinoic acid) and 10% urea, and the treatment of hyperkeratoses with a combination of 10% urea and 10% salicylate.

Beastall et al. describe a study in which a decrease in the time taken to induce erythema by topically applied nicotinate was observed as the nicotinate was combined with increasing amounts of urea.

U.S. Pat. No. 5,879,690 (Perricone et al.) describes the use of catecholamines and related compounds in combination with percutaneous penetration enhancers for topical administration of sagging subcutaneous muscle. The use of several enhancers is suggested, including urea, as is the inclusion of compounds that scavenge free radicals and anti-oxidants, for example, vitamins E and C. While the use of enhancers up to a concentration of about 10% is suggested, the teachings in regard to enhancers are quite general and there does not appear to be any suggestion that the use of urea would produce any benefits beyond those known in the art at the time.

There are commercially available skin moisturizers that contain up to about 25% urea for aid in softening and hydrating hard dry skin.

WO 86/00014 (Weiner), published Jan. 3, 1986, describes topical cream compositions that includes 15% urea, possibly up to 40% urea, in combination with UV absorbing sun screen agents, for the purposes of prevention and/or reduction of skin damage caused by reactive chemical substances generated in the skin by ultraviolet radiation.

In the context of this invention, the amount of vitamin A to be included in a composition would be from about 0.1 to about 5%, vitamin E would be from about 0.1 to about 10%.

In vivo efficacy can be evaluated against dry skin in the case of active ingredients that would be thought to be helpful to such condition. Subjects with dry itchy skin use a product containing urea in combination with the test ingredient in a controlled clinical test. The skin is graded, subjective assessments are obtained and a number of different instruments are used to determine effects on skin. Again systematic studies using various concentrations of urea, various concentrations of the test ingredient, test times and controls (compositions lacking the test ingredient and/or urea, for example) are carried out to determine optimal composition makeup.

In vivo barrier repair can be tested for ingredients thought to play a role in wound repair. Tape stripping, for example, is used to damage skin and the damage is then judged by measuring trans-epidermal water loss and erythema. Test compositions can be applied to the damage area and effects on the rate of healing determined. Again, various concentrations of the test ingredient, in combination with various concentrations of urea, and appropriate controls, etc., would be used. Additionally, of course, different application methods might also be tested.

Protection against UV damage can also be studies in in vivo experiments. Vitamins E and C, especially in combination with each other, have been shown to protect against sunburn even though they do not absorb significant amounts of UV light. This is because they reduce damage caused by free radicals that result from UV exposure.

In terms of the minimum amount of urea that is to be included in a composition in combination with a particular active ingredient, it may be found that there is a particular minimum associated with a group of ingredients that are chemically related to the one tested. For example, if it is found that at least 20% urea is suitable for two or more compounds of a family of compounds that a person skilled in the art would understand to share hydrophilic properties, have similar molecular weights, etc., then it would be reasonably expected that 20% urea would be a suitable amount of urea to be used in combination with other compounds of that family. Of course, the greater the number of compounds within a family that are actually tested and found to behave similarly, the greater the certainty that other compounds of the family that are not tested will behave similarly to those that have been tested.

Various active ingredients that can be included in compositions of the present invention, alone or in combination, are described below.

Allopurinol is not, strictly speaking, an antioxidant. This ingredient is though to suppress the body's production of an oxidation catalyst, xanthine oxidase.

Alpha lipoic acid (ALA) is a water soluble and lipid soluble antioxidant. Apparently, ALA promtes the regeneration of the redox reaction between vitamin C, E, and glutathione.⁵⁸

Astaxanthin is a carotenoid produced by a microalgae called Haematocococcus pluvialis.

Azulenic retinoid compounds are compounds related to Vitamin A and retinoic acid. A new type of azulene-containing retinoid has been synthesised and is similar in size and shape to Vitamin A, but its electronic properties are different.

Vitamin A (β-carotene, carotenoid, lycopene, xanthophylls and lycopene) is a term loosely used to describe members of a family of anti-oxidant substances called carotenoids. These carotenoids have antioxidant and other qualities and can be converted by the body into vitamin A.

Vitamin C (ascorbic acid) must be obtained from food or vitamin supplements. This anti oxidant is thought to slow down loss of glutathione to neutralise some destructive cell oxidants.

Vitamin E occurs in nature in several forms—alpha, beta, delta, and gamma-tocopherols and alpha, beta, delta and gamma tocotrienols. Most vitamin E supplements contain alpha tocopherol form which is thought to have significant biological activity.⁵⁹

Melatonin has been found to rescue DA neurons from cell death in several experimental paradigms associated with oxidative stress.⁶⁰ The combined findings suggest that melatonin counteracts the in vitro destructive effects of NMDA or hypoxia/reperfusion by preventing accumulation of excessive free radicals.⁶¹ Melatonin protects primary cultures of rat cortical neurones from NMDA excitotoxicity and hypoxia/reoxygenation.⁶²

Silica hydride. This silica mineral is 5 nanometers in total area—the smallest nutritional particle ever discovered. When combined, this molecule is a million times smaller than the next smallest antioxidant.

N-Acetylcysteine (NAC) can be derived from the amino acid cysteine. NAC is a natural sulfur-containing amino acid derivative found naturally in foods and is thought to have antioxidant properties.⁶³

Phenolics are naturally occurring anti oxidants found in the skins of many fruits, vegetables and herbs.

Pyrimidines are a group of antioxidant compounds, the pyrrolopyrimidines, discovered recently. They appear to quench lipid peroxidation reactions by electron-donating and/or radical-trapping mechanisms.⁶⁴

Activin (TM) can be obtained from the seeds of the red grape.

Superoxide Dimutase (SOD) is available in oral an form called Prozyme (or Polbax in Sweden).⁶⁵

Black and green teas (camelia sinensis) have anti oxidant properties. Black tea is though to have similar anti oxidant properties to those of green tea.

OPC or proanthocynidins. (Pycnogenol (grape seed extract) is described in U.S. Pat. No. 4,698,360).⁶⁶

Coenzyme Q-10 (ubiquinone) is an essential cofactor of the electron transport chain as well as a potent free radical scavenger in lipid and mitochondrial membranes.⁶⁷ Coenzyme Q10 administration is though to increase brain mitochondrial concentrations and to exert neuroprotective effects.⁶⁸

There are many examples of herbal antioxidants. For example, silymarin, the flavonoid complex of milk thistle (Silybum marianum) and Ginkgo biloba extract is thought to be an antioxidant.

Other herbal antioxidants include Silymarin (liver); Ginkgo biloba (brain and circulation); pycnogenols (veins); and bilberry (retina).

Curcumin can be obtained from tumeric. Curcumin is the yellow pigment of turmeric (Curcuma longa), an ingredient of curry powder and prepared mustard. Curcumin is though to be an antioxidant.

The amount of active ingredient to be included in a composition is a topically effective amount, and can be determined by a person skilled in the art according to the purposes for which the ingredient is being applied. Thus, for example, in the foregoing example involving Composition E, it was found that the amount of vitamin E measured at the skin level just above the viable epidermis is between two and three times that found when no urea was used (control). Thus the amount of vitamin E to be included in a composition containing 25% urea can be ⅓ to ½ the amount that would be included in a composition lacking urea. Of course, a person skilled in the art, provided with this specification would be readily capable of deriving other formulations within the scope of the invention described herein.

This application claims priority from U.S. Provisional Patent Application Serial No. 60/152,637 filed Sep. 7, 1999, the contents of which are incorporated herein by reference.

All references cited herein are incorporated into this document in their entirety by reference thereto.

### REFERENCES

1. Thiele, J. J., Traber, M. G., & Packer, L. (1998) J Invest Dermatol 110, 756.

2. Alster, T. S. & West, T. B. (1998) Dermatol Surg 24, 331.

3. Saliou, C., Kitazawa, M., McLaughlin, L., Yang, J. P., Lodge, J. K., Tetsuka, T., Iwasaki, K., Cillard, J., Okamoto, T., & Packer, L. (1999) Free Radic Biol Med 26, 174.

4. Wolf, R., Wolf, D., & Ruocco, V. (1998) J Eur Acad Dermatol Venereol 10, 103.

5. Shukla, A., Rasik, A. M., & Patnaik, G. K. (1997) Free Radic Res 26, 93.

6. Fortune Magazine, Jul. 5, 1999, 144

7. Packer L., Feb. 26, 1996 address to Oxygen Club of CA

8. Steenvoorden, D. P. & van Henegouwen, G. M. (1997) J Photochem Photobiol B 41, 1.

9. Han, F. N., Hart J 1995, Oats: Chemistry, Technology & Potential Uses . . . , C&T March 110, 63

10. Tajima, S. & Pinnell, S. R. (1996) J Dermatol Sci 11, 250.

11. Phillips, C. L., Combs, S. B., & Pinnell, S. R. (1994) J Invest Dermatol 103, 228.

12. Ponec, M., Weerheim, A., Kempenaar, J., Mulder, A., Gooris, G. S., Bouwstra, J., & Mommaas, A. M. (1997) J Invest Dermatol 109, 348.

13. Alster, T. S. & West, T. B. (1998) Dermatol Surg 24, 331.

14. McVean, M. & Liebler, D. C. (1999) Mol Carcinog 24, 169.

15. McVean, M. & Liebler, D. C. (1997) Carcinogenesis 18, 1617. McVean, M. & Liebler, D. C. (1997) Carcinogenesis 18, 1617.

16. Nachbar, F. & Korting, H. C. (1995) J Mol Med 73, 7.

17. Young, K. J. & Lee, P. N. (1999) Eur J Cancer Prev 8, 91.

18. Simon, G. A., Schmid, P., Reifenrath, W. G., van Ravenswaay, T., & Stuck, B. E. (1994) J Pharm Sci 83, 1101.

19. Nachbar, F. & Korting, H. C. (1995) J Mol Med 73, 7.

20. Gehring, W., Fluhr, J., & Gloor, M. (1998) Arzneimittelforschung 48, 772.

21. Darr et al., Acta Derm Venerol (Stockholm) 1996; 76 264-268.

22. Pinnell.

23. Goffin, V., Henry, F., Pierard-Franchimont, C., & Pierard, G. E. (1997) Skin Pharmacol 10, 85.

24. Campos, P., Eccleston, G. 1998, Vitamin A Skin Pennetration C&T July 113, 69

25. C. G. Fthenakis, D. H. Maes and W. P. Smith, In vivo assement of skin elasticity using ballistometry, J. Soc. Cosmet. Chem 42, 211-222(1991).

26. Fox, C., C&T March, 114 No2, 22

27. Niwa, Y. (1999) Rinsho Byori 47, 189.

28. Goffin, V., Henry, F., Pierard-Franchimont, C., & Pierard, G. E. (1997) Skin Pharmacol 10, 85.

29. McVean, M. & Liebler, D. C. (1999) Mol Carcinog 24, 169.

30. Nachbar, F. & Korting, H. C. (1995) J Mol Med 73, 7.

31. Bekyarova, G. & Yankova, T. (1998) Acta Physiol Pharmacol Bulg 23, 55.

32. Dreher, F., Denig, N., Gabard, B., Schwindt, D. A., & Maibach, H. I. (1999) Dermatology 198, 52.

33. Gensler, H. L., Aickin, M., Peng, Y. M., & Xu, M. (1996) Nutr Cancer 26, 183.

34. Darr, D., Dunston, S., Faust, H., & Pinnell, S. (1996) Acta Derm Venereol 76, 264.

35. Werninghaus, K., Meydani, M., Bhawan, J., Margolis, R., Blumberg, J. B., & Gilchrest, B. A. (1994) Arch Dermatol 130, 1257.

36. Pinnell S., Duke University, Interviewed by Norm Swan Jun. 30, 1997

37. Gupta, S., Ahmad, N., Mohan, R. R., Husain, M. M., & Mukhtar, H. (1999) Cancer Res 59, 2115.

38. Loncar, Clifford, March 1996, happi, 85

39. Noda, Y., Anzai, K., Mori, A., Kohno, M., Shinmei, M., & Packer, L. (1997) Biochem Mol Biol Int 42, 35.

40. Katiyar, S. K. & Mukhtar, H. (1997) J Cell Biochem Suppl 27, 59.

41. Katiyar, S. K., Mohan, R. R., Agarwal, R., & Mukhtar, H. (1997) Carcinogenesis 18, 497.

42. Mukhtar, H., Katiyar, S. K., & Agarwal, R. (1994) J Invest Dermatol 102, 3.

43. Hassan Muktar.

44. Mansell, P. W. A., 1994 Polysaccharides in skin care, C&T, Sept., 109, 67

45. Ji, H. and Seo B., 1999, Retinyl Palmitate at 5% . . . C&T March, 114, 61

46. Darr, D., Dunston, S., Faust, H. and Pinnell, S. (1996) Effectiveness of antioxidants (vitamin C and E) with and without sunscreens as topical photoprotectants. Acta Derm Venereol 76, 264-268.

47. Van Duzee, (J Invenst Dermatol 71:140-144, 1978)

48. W. Wohlrab, “Use and efficacy of urea in dermatological preparations” Presented at the 3rd International Congress on Cosmetic Dermatology “progress in Cosmetic Dermatology”, October 1989. Wien.

49. Raab, W. P., 1990, Allergy Clinic, Vienna Austria, C&T., 104

50. Collett, J. H., Flood B, L 1976, Some effects of Urea on Drug Dissolution, J. Pharm. Pharmac., 28, 206-209

51. Simon, G. A., Schmid, P., Reifenrath, W. G., van Ravenswaay, T., & Stuck, B. E. (1994) J Pharm Sci 83, 1101.

52. U.S. Pat. No. 5,353,802, issued Oct. 11, 1994.

53. “Instrument evaluation of skin irritation”, P. Y. Rizvi, B. M. Morrison, Jr., M. J. Grove and G. L. Grove, Cosmetics & Toiletries., 111: 39, 1996.

54. “Electrical impedance index in human skin: Measurements after occlusion, in 5 anatomical regions and in mild irritant contact dermatitis”, L. Emtestam and S. Ollmar, Cont. Derm. 28: 337, 1975.

55. “Electrical impedance for estimation of irritation in oral mucosa and skin”, S. Ollmar, E. Eek, F. Sundstrom and L. Emtestam, Medical Progress Through Technology, 21: 29, 1995.

56. “Electrical impedance compared with other non-invasive bioengineering techniques and visual scoring for detection of irritation in human skin”, S. Ollmar, M. Nyren, I. Nicander and L. Emtestam, Brit. J. Dermatol. 130: 29, 1994.

57. “Correlation of impedance response patterns to histological findings in irritant skin reactions induced by various surfactants”, I. Nicander, S. Ollmar, A. Eek, B. Lundh Rozell and L. Emtestam, Brit. J. Dermatol. 134: 221, 1996.

58. Packer, Lester, Ph.D., et al. “Alpha-Llpoic Acid As A Biological Antioxidant,” Free Radical Boilogy and Medicine 19:227-250, 1995. Also—Passwater Richard A., Ph.D. Lipoic Acid: The Metabolic Antioxidant. New Canaan, Conn. Keats Publishing, Inc., 1995 pp. 7-8.

59. Christen S., et al., Gamma-tocopherol traps mutagenic electrophiles such as NOx and complements alpha-tocopherol: Physiological implications. Proc. Natl. Acad. Sci. USA, vol 94, pp. 3217-3222, April 1997.

60. February 98, Neuropathy Digest “Melatonin rescues dopamine neurons from cell death in tissue culture models of oxidative stress”. Iacovitti L, Stull N D, Johnston K. Department of Neurobiology and Anatomy, Allegheny University of the Health Sciences, Philadelphia, Pa. 19102, USA.

61. Brain Res Sep. 12, 1997; 768(1-2):120-124

62. Cazevieille C, Safa R, Osborne N N Nuffield Laboratory of Ophthalmology, Oxford University, UK.

63. Louwerse, E. S., et al. JO: *Archives of Neurology* v52, p559-564, June 1995.

64. Excerpt from AU: Hall E D; Andrus P K, Smith S L; Fleck T J; Scherch H M; Lutzke B S; Sawada G A; Althaus J S; Vonvoigtlander P F; Padbury G E; Larson P G; Palmer J R; Bundy G L TI: Pyrrolopyrimidines: novel brain-penetrating antioxidants with neuroprotective activity in brain injury and ischemia models. SO: JOURNAL OF PHARMACOLOGY AND EXPERIMENTAL THERAPEUTICS, 281 (2) 895-904/1997 May).

65. Per Christer Odén, Department of Forest Genetics and Plant Physiology, Swedish University of Agricultural Sciences, S-901 83 Umeå, Sweden and Roland Einarsson, Pharmacia Diagnostics, S-751 82 Uppsala, Sweden. Rabinowitch and Fridovich 1983, Fridovich 1986, Asada 1988, Monk et al. 1989, Hassan and Scandalios 1990.

66. “The New Superantioxidant-Plus” by Richard A. Passwater Ph.D. (Keats Publishing, Inc., New Canaan, Conn.).

67. Proc Natl Acad Sci USA Jul. 21, 1998; 95(15):8892-8897

68. Matthews R T, Yang L, Browne S, Baik M, Beal M F Neurochemistry Laboratory, Neurology Service, Massachusetts General Hospital and Harvard Medical School, Boston, Mass. 02114, USA.

1. A topical composition comprising:
about 15 to about 50% by weight urea with respect to the total composition weight of the composition; and
a topically effective amount of vitamin E, vitamin C, and an anti-oxidant compatible with skin, wherein the anti-oxidant is selected from the group consisting of retinyl palmitate, β-carotene, green tea, black tea, and combinations thereof.

2. The composition of claim 1, wherein the composition further comprises a topically effective amount of at least one of the following: vitamin D, quercetin, sea kelp, pycnogenols (proanthocyanidins), selenium and alkylgylcerol-AKG, allopurinol, α-lipoic acid, astaxanthin, azulenic retinoid compounds, coenzyme Q-10, cysteine, zinc, copper, magnesium, potassium, selenium, BHA, BHT, melatonin, and N-acetylcysteine.

3. The composition of claim 1, comprising between about 20 and 40% by weight urea with respect to the total composition weight of the composition.

4. The composition of claim 2, comprising between about 20 and 40% by weight urea with respect to the total composition weight of the composition.

5. A topical composition comprising:
about 20 to about 40% by weight urea with respect to the total composition weight of the composition; and
a topically effective amount of vitamin E.

6. The composition of claim 5, wherein the composition further comprises a topically effective amount of at least one of the following: vitamin C, retinyl palmitate, β-carotene, green tea, black tea, vitamin D, quercetin, sea kelp, pycnogenols (proanthocyanidins), selenium and alkylgylcerol-AKG, allopurinol, α-lipoic acid, astaxanthin, azulenic retinoid compounds, coenzyme Q-10, cysteine, zinc, copper, magnesium, potassium, selenium, BHA, BHT, melatonin, and N-acetylcysteine.

7. The composition of claim 6, comprising between about 20% and about 35% urea.

8. The composition of claim 7, comprising about 25% urea.

9. The composition of claim 5, wherein the vitamin E is present as tocopherol acetate.

10. The composition of claim 5, wherein the composition comprises between about 0.1% and about 10% by weight of vitamin E.

11. The composition of claim 10, wherein the composition comprises at least about 0.5% by weight of vitamin E.

12. The composition of claim 11, wherein the composition comprises between about 1% and 2% by weight of vitamin E.

13. The composition of claim 5, wherein the composition includes green tea extract.

14. The composition of claim 20, comprising vitamin A, vitamin C, vitamin E and green tea extract.

15. A method of enhancing delivery of an anti-oxidant to the viable epidermis, the method comprising the step of applying to the skin surface a composition comprising at least 10% urea and a topically effective amount of vitamin E.

16. The method of claim 15, wherein the composition comprises at least 15% urea.

17. The method of claim 16, wherein the composition comprises at least 20% urea.

18. The method of claim 17, wherein the composition comprises at least 25% urea.

19. A method of enhancing delivery of an anti-oxidant to the viable epidermis, the method comprising the step of applying to the skin surface of a mammal a composition according to claim 1.

20. A method of enhancing delivery of an anti-oxidant to the viable epidermis, the method comprising the step of applying to the skin surface of a mammal a composition according to claim 2.

21. A method of enhancing delivery of an anti-oxidant to the viable epidermis, the method comprising the step of applying to the skin surface of a mammal a composition according to claim 3.

22. A method of enhancing delivery of an anti-oxidant to the viable epidermis, the method comprising the step of applying to the skin surface of a mammal a composition according to claim 4.

23. A method of enhancing delivery of an anti-oxidant to the viable epidermis, the method comprising the step of applying to the skin surface of a mammal a composition according to claim 5.

24. A method of enhancing delivery of an anti-oxidant to the viable epidermis, the method comprising the step of applying to the skin surface of a mammal a composition according to claim 6.

25. A method of enhancing delivery of an anti-oxidant to the viable epidermis, the method comprising the step of applying to the skin surface of a mammal a composition according to claim 7.

26. A method of enhancing delivery of an anti-oxidant to the viable epidermis, the method comprising the step of applying to the skin surface of a mammal a composition according to claim 8.

27. A method of enhancing delivery of an anti-oxidant to the viable epidermis, the method comprising the step of applying to the skin surface of a mammal a composition according to claim 9.

28. A method of enhancing delivery of an anti-oxidant to the viable epidermis, the method comprising the step of applying to the skin surface of a mammal a composition according to claim 10.

29. A method of enhancing delivery of an anti-oxidant to the viable epidermis, the method comprising the step of applying to the skin surface of a mammal a composition according to claim 14.

30. A method of manufacturing a topical preparation comprising a composition of claim 1, the method comprising combining the components of said composition so as to form a homogeneous topical skin preparation.

31. A method of manufacturing a topical preparation comprising a composition of claim 2, the method comprising combining the components of said composition so as to form a homogeneous topical skin preparation.

32. A method of manufacturing a topical preparation comprising a composition of claim 3, the method comprising combining the components of said composition so as to form a homogeneous topical skin preparation.

33. A method of manufacturing a topical preparation comprising a composition of claim 4, the method comprising combining the components of said composition so as to form a homogeneous topical skin preparation.

34. A method of manufacturing a topical preparation comprising a composition of claim 5, the method comprising combining the components of said composition so as to form a homogeneous topical skin preparation.

35. A method of manufacturing a topical preparation comprising a composition of claim 6, the method comprising combining the components of said composition so as to form a homogeneous topical skin preparation.

36. A method of manufacturing a topical preparation comprising a composition of claim 7, the method comprising combining the components of said composition so as to form a homogeneous topical skin preparation.

37. A method of manufacturing a topical preparation comprising a composition of claim 8, the method comprising combining the components of said composition so as to form a homogeneous topical skin preparation.

38. A method of manufacturing a topical preparation comprising a composition of claim 9, the method comprising combining the components of said composition so as to form a homogeneous topical skin preparation.

39. A method of manufacturing a topical preparation comprising a composition of claim 10, the method comprising combining the components of said composition so as to form a homogeneous topical skin preparation.

40. A method of manufacturing a topical preparation comprising a composition of claim 14, the method comprising combining the components of said composition so as to form a homogeneous topical skin preparation.

41. A method according to claim 30, wherein the skin preparation is a cosmetic preparation.

42. A method according to claim 30, wherein the skin preparation is a cosmetic preparation.

43. A method according to claim 30, wherein the skin preparation is a cosmetic preparation.

44. A method according to claim 30, wherein the skin preparation is a cosmetic preparation.

45. A method according to claim 30, wherein the skin preparation is a cosmetic preparation.

46. A method according to claim 30, wherein the skin preparation is a cosmetic preparation.

47. A method according to claim 30, wherein the skin preparation is a cosmetic preparation.

48. A method according to claim 30, wherein the skin preparation is a cosmetic preparation.

49. A method according to claim 30, wherein the skin preparation is a cosmetic preparation.

50. A method according to claim 30, wherein the skin preparation is a cosmetic preparation.

51. A method according to claim 30, wherein the skin preparation is a cosmetic preparation.
